(19) United States
(12) Patent Application Publication
(10) Pub. No.: US 2023/0190725 A1
(43) Pub. Date: Jun. 22, 2023

(54) MEK INHIBITORS FOR CORNEAL SCARRING AND NEOVASCULARIZATION

(71) Applicant: THE BOARD OF TRUSTEES OF THE UNIVERSITY OF ILLINOIS, Urbana, IL (US)

(72) Inventor: Ali R. Djalilian, Chicago, IL (US)

(21) Appl. No.: 17/511,635

(22) PCT Filed: Apr. 27, 2020
(86) PCT No.: PCT/US2020/030150

§ 371 Date: Oct. 27, 2021

Related U.S. Application Data
(60) Provisional application No. 62/840,069, filed on Apr. 29, 2019.

(51) Int. Cl. A61K 31/4523; A61P 27/02; A61K 47/20; A61K 47/38; A61K 31/166; A61K 31/18; A61K 9/00

(52) U.S. Cl. CPC A61K 31/4523; A61P 27/02; A61K 47/20; A61K 47/38; A61K 31/166; A61K 31/18; A61K 9/0053; A61K 9/0048

(57) ABSTRACT

The present disclosure relates to ophthalmic compositions and methods for inducing PAX6 expression in a cells and for reducing or preventing corneal scarring, corneal neovascularization and/or corneal opacification. The disclosure also provide for ophthalmic compositions and methods for improving vision or reducing limitations on vision.

### CROSS REFERENCE TO RELATED APPLICATIONS

This application claims priority benefit of U.S. Provisional Application No. 62/840,069, filed on Apr. 29, 2019, which is incorporated by reference herein in its entirety.

### STATEMENT REGARDING FEDERALLY FUNDED RESEARCH

The present disclosure was made with government support under grant number 5R01 EY024349 awarded by the National Eye Institute. The government has certain rights in the disclosure.

### FIELD OF INVENTION

The present disclosure relates to ophthalmic compositions and methods for inducing PAX6 expression in a cells and for reducing or preventing scarring, neovascularization and/or opacification on the surface of the eye. The disclosure also provide for ophthalmic compositions and methods for improving vision or reducing limitations on vision.

### BACKGROUND OF THE DISCLOSURE

The surface of the eye is made up of cornea and the clear membrane called the conjunctiva. The conjunctiva is connective tissue that covers the surface of the eye and forms the inner surface of the eyelid. The conjunctiva adheres to the sclera at the limbus to meet the cornea. The cornea is the clear outer layer at the front of the eye, which helps the eye focus light. Small injuries, such as scratches, on the cornea usually heal on its own but deeper scratches or other injuries, such as abrasions, lacerations, burns or disease, can cause corneal scarring and vision problems. Depending on the degree of corneal scarring, vision can range from a blur to total blindness. In particular, these deeper injuries result in loss of corneal tissue, which is replaced by scar tissue that is protected by fibroblast cells. In addition, scarring from disease, such as due to infections or inflammation, is usually the result of growth of new blood vessels (neovascularization) into the clear cornea. Disease that cause neovascularization include herpes simplex, herpes zoster, syphilis, bacterial, fungal and other immune mediated keratitis.

Mitogen-activated protein kinase (MEK1/MEK2) is a kinase in the Ras-MAPK pathway which phosphorylates and activates MAPK (mitogen-activated protein kinase). Overactivation of the Ras-MAPK signaling cascade has been implicated in the development of malignancies, such as melanoma. Mapk/ERK kinase (MEK) inhibitors are anti-proliferative medications widely used in the clinic to limit progression of several neoplastic diseases, and they have been shown to reduce VEGF pathway stimulation.

Pax6 is one of the major regulators of eye development and is important in tissue regeneration. The human paired box 6 (PAX6), is a member of the PAX family of transcription factors, and is known to be one of the major regulators of eye development and is important in tissue regeneration. During embryonic development, the PAX6 protein is thought activate genes involved in the formation of the eyes, the brain and spinal cord (central nervous system), and the pancreas. Within the brain, the PAX6 protein is involved in the development of a specialized group of brain cells that process smell (the olfactory bulb). Additionally, researchers believe that the PAX6 protein controls many aspects of eye development before birth. After birth, the PAX6 protein likely regulates the expression of various genes in many structures of the eyes.

There is a need for treatments which reduce the progression of corneal scarring. MEK pathway is known to inhibit the PAX6 pathway. The data provided herein demonstrates that MEK inhibitors stimulate PAX6 and thereby reduce corneal scarring.

### SUMMARY OF INVENTION

The present disclosure is directed to a topical medication therapy for preventing scarring and neovascularization on the surface of the eye Some examples of the diseases and conditions for this therapy include ocular surface inflammation, severe ocular surface injuries, limbal stem cell deficiency, cancers, inflammation, infection as well as congenital diseases. It is well known that scarring is partially due to fibroblasts' reproduction and maldifferentiation. It has been also shown that neovascularization is partially due to VEGF pathway stimulation in inflammatory conditions following injury.

The studies described herein demonstrate that several MEK inhibitors induce PAX6 production to a desirable extent in vitro in human corneal epithelial cells. In particular, MEK inhibitor, PD0325901, a potent MEK inhibitor was tested in an epithelial and limbal stem cell injury model, in which the human corneal epithelial cells (HCEC) and in the epithelium of the cornea up to the limbal area was injured creating a severe scar forming wound with limbal stem cell deficiency in a mouse model of Aniridia.

The disclosure provides for ophthalmic composition comprising a MAPK/ERK kinase (MEK) inhibitor and phosphate buffered saline (PBS). In some embodiments, the ophthalmic composition comprises about 0.1 to about 10 μM MEK inhibitor, about 1 to about 20% dimethyl sulfoxide (DMSO) and about 1 to about 20% hydroxypropyl methylcellulose (HPMC). In another embodiment, the ophthalmic composition comprises about 0.1 to about 10 μM MEK inhibitor, about 10% to about 50% poloxamer, such as poloxamer 407 (also known as pluronic F127). In particular embodiments, the MEK inhibitor is PD0325901 or mirdametinib. An exemplary composition is about 1 μm PD0325901, about 2% DMSO, about 20% HPMC, in PBS. Another exemplary composition is about 1 μm PD0325901, about 30% poloxamer 407, in PBS.

In exemplary embodiments, the MEK inhibitor is in a concentration ranging from about 0.1 to about 10 μM MEK inhibitor, or about 0.2 to about 20 μM MEK inhibitor, or about 0.05 to about 50 μM MEK inhibitor, or 0.1 to about 5 μM MEK inhibitor, or 0.1 to about 1 μM MEK inhibitor, or about 0.25 to about 8 μM MEK inhibitor, or about 0.25 to about 6 μM MEK inhibitor, or 0.25 to about 5 μM MEK inhibitor, or about 0.25 to about 3 μM MEK inhibitor, or about 0.25 to about 1 μM MEK inhibitor, or about 0.5 to about 20 μM MEK inhibitor, or about 0.5 to about 10 μM MEK inhibitor, or about 0.5 to about 8 μM MEK inhibitor, or 0.5 to about 5 μM MEK inhibitor, or 0.5 to about 3 μM MEK inhibitor, or 0.5 to about 1 μM MEK inhibitor, or about 1 to about 5 μM MEK inhibitor, or about 1 to about 10 μM MEK inhibitor, or about 1 to about 20 μM MEK inhibitor, or 1 to about 50 μM MEK inhibitor, or 5 to about 10 μM MEK inhibitor, or 5 to about 20 μM MEK inhibitor. Exemplary concentrations of MEK inhibitor in the disclosed compositions include about 0.1 μM MEK inhibitor, about 0.2 μM MEK inhibitor, about 0.3 μM MEK inhibitor, about 0.4 μM MEK inhibitor, about 0.5 μM MEK inhibitor, about 0.6 μM MEK inhibitor, about 0.7 μM MEK inhibitor, or about 0.8 μM MEK inhibitor, or about 0.9 μM MEK inhibitor, about 1 μM MEK inhibitor, about 2 μM MEK inhibitor, about 3 μM MEK inhibitor, about 4 μM MEK inhibitor, about 5 μM MEK inhibitor, about 6 μM MEK inhibitor, about 7 μM MEK inhibitor, about 8 μM MEK inhibitor, about 9 μM MEK inhibitor, about 10 μM MEK inhibitor, about 12 μM MEK inhibitor, about 15 μM MEK inhibitor, about 18 μM MEK inhibitor, about 20 μM MEK inhibitor, about 30 μM MEK inhibitor, about 40 μM MEK inhibitor or about 50 μM MEK inhibitor.

In some embodiments, the MEK inhibitor is a MEK1 or MEK2 inhibitor, such as PD098059, PD0325901/Mirdametinib, Trametinib, Cobimetinib, MEK 1/2 inhibitor AS703988, MEK inhibitor AZD8330, MEK inhibitor CS3006, MEK inhibitor RO4987655, MEK inhibitor SHR 7390, MEK inhibitor TAK-733, MEK/Aurora kinase dual inhibitor BI 847325, MEK-1/MEKK-1 inhibitor E6201, refametinib, selumetinib, binimetinib, CI-1040, AZD6244, GDC-0973, GSK1120212, AS703026, BS203580, MEK162, GDC-0623, RO5126766, WX-554, HL-085, or U0126.

In some embodiments, the MEK inhibitor is an ERK inhibitor, such as is FRI-20, ON-01060, VTX-11e, 25-OH-D3-3-BE, B3CD, bromoacetoxycalcidiol, 180204 AEZ-131, AEZS-131, AEZS-136, SCH-772984, AZ-13767370, BL-EI-001, LY-3214996, LTT-462, KO-947 CC-90003, GDC-0994, RG-7842, MK-8353, SCH900353, BVD-523, or Ulixertinib.

In some embodiments, the composition comprises about 1% to about 20% DMSO, or about 1% to about 20% DMSO, 2% to about 30% DMSO, or about 3% to about 40% DMSO, or about 4% to about 50% DMSO, or about 1% to about 10% DMSO, or about 1% to about 5% DMSO, or about 2% to about 20% DMSO, or about 2% to about 10% DMSO, or about 2% to about 5% DMSO, or about 5% to about 10% DMSO, or about 5% to about 20% DMSO, or about 5% to about 50% DMSO, or about 10% to about 20% DMSO, or about 12% to about 20% DMSO, or about 15% to about 20% DMSO, or about 20% to about 50% DMSO. Exemplary DMSO concentration include 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40% or 50%.

In addition, the disclosed compositions comprise about 1% to about 20% HPMC, or about 1% to about 20% HPMC, 2% to about 30% HPMC, or about 3% to about 40% HPMC, or about 4% to about 50% HPMC, or about 1% to about 10% HPMC, or about 1% to about 5% HPMC, or about 2% to about 20% HPMC, or about 2% to about 10% HPMC, or about 2% to about 5% HPMC, or about 5% to about 10% HPMC, or about 5% to about 20% HPMC, or about 5% to about 50% HPMC, or about 10% to about 20% HPMC, or about 12% to about 20% HPMC, or about 15% to about 20% HPMC, or about 20% to about 50% HPMC. Exemplary HPMC concentration include 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40% or 50%.

In addition, the disclosed compositions comprises a poloxamer, such as poloxamer 407 (also known as pluronic F127). For example, the disclosed comprises about 1% to about 20% poloxamer, or about 1% to about 20% poloxamer, 2% to about 30% poloxamer, or about 3% to about 40% poloxamer, or about 4% to about 50% poloxamer, or about 1% to about 10% poloxamer, or about 1% to about 5% poloxamer, or about 2% to about 20% poloxamer, or about 2% to about 10% poloxamer, or about 2% to about 5% poloxamer, or about 5% to about 10% poloxamer, or about 5% to about 20% poloxamer, or about 5% to about 50% poloxamer, or about 10% to about 20% poloxamer, or about 12% to about 20% poloxamer, or about 15% to about 20% poloxamer, or about 20% to about 50% poloxamer. Exemplary poloxamer concentrations include 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40% or 50%.

In various aspects, the disclosed ophthalmic composition comprise one or more of pharmaceutically acceptable ophthalmic excipients. For example, the pharmaceutically acceptable ophthalmic excipients is selected from Cyclodextrins, Carbopol or carbomer or acrylic acid polymers, Poloxamers, Xyloglucan, Methylcellulose, Hydroxypropyl Methylcellulose, Ethyl (Hydroxyethyl) Cellulose, Pseudolatexes, Cellulose Acetate Phthalate, Gellan Gum, Alginate, Carrageenans, Hyaluronic Acid, Sodium acetate, Edetate disodium, Hypromellose, Acetic acid, Alcohol, Alginic acid, Amerchol-cab, Antipyrine, Benzalkonium chloride, Benzododecinium bromide, Boric acid, Caffeine, Calcium chloride, Carbomer 1342, Carbomer 934P, Carbomer 940, Carbomer homopolymer type B (allyl pentaerythritol cross-linked), Carboxymethylcellulose sodium, Castor oil, Cetyl alcohol, Chlorobutanol, Citric acid, Citric acid monohydrate, Creatinine, Divinylbenzene styrene copolymer, Ethylene vinyl acetate copolymer, Gellan gum (low acyl), Glycerin, Glyceryl stearate, Hypromelloses, Lanolin, Lauralkonium chloride, Lauroyl sarcosine, Magnesium chloride, Methylparaben, Mineral oil, Nonoxynol-9, Octoxynol-40, Petrolatum, Phenylethyl alcohol, Phenylmercuric acetate, Phenylmercuric nitrate, Polidronium chloride, Poloxamer 188 or 407, Polycarbophil, Polyethylene glycol 400 or 8000, Polyoxyl 35 castor oil, Polyoxyl 40 hydrogenated castor oil, Polyoxyl 40 stearate, Polypropylene glycol, Polysorbate 20, Polyvinyl alcohol, Potassium chloride, Potassium sorbate, Povidone K29/32, Povidone K30, Povidone K90, Povidones, Propylene glycol, Propylparaben, Soda ash, Sodium acetate, Sodium bisulfate, Sodium borate, Sodium borate decahydrate, Sodium carbonate, Sodium chloride, Sodium citrate, Sodium metabisulfite, Sodium nitrate, Sodium sulfate, Sodium sulfite, Sodium thiosulfate, Sorbic acid, Sorbitol, Stabilized oxychloro complex, Sulfuric acid, Thimerosal, Titanium dioxide, Tocophersolan, Trisodium citrate dehydrate, Tromethamine, Tyloxapol, Xanthan gum, Zinc chloride, or a combination thereof.

In exemplary embodiments, the ophthalmic composition is provided in a suitable carrier and/or is formulated for topical administration.

The disclosure provides for method of inducing PAX6 expression in a cell comprising administering to the cell an effective amount of a MAPK/ERK kinase (MEK) inhibitor. For example, the cell is an epithelial cell or a fibroblast cell on the surface of the eye, such as a corneal epithelial cell, a conjunctiva epithelia cell, a corneal fibroblast cell or a conjunctiva fibroblast cell.

The disclosure also provides for use of the an effective amount of a MAPK/ERK kinase (MEK) inhibitor for the preparation of a medicament for the inducing PAX6 expression in a cell. For example, the cell is an epithelial cell or a fibroblast cell on the surface of the eye, such as a corneal epithelial cell, a conjunctiva epithelia cell, a corneal fibroblast cell or a conjunctiva fibroblast cell.

In addition, the disclosure provides for a composition for inducing PAX6 expression in a cell, wherein the composition comprises an effective amount of a MAPK/ERK kinase (MEK) inhibitor. For example, the cell is an epithelial cell or a fibroblast cell on the surface of the eye, such as a corneal epithelial cell, a conjunctiva epithelia cell, a corneal fibroblast cell or a conjunctiva fibroblast cell.

In another embodiment, the disclosure provides for methods of reducing or preventing scarring, opacification, or neovascularization in the cornea or the surface of the eye of a subject comprising administering to the subject an effective amount of a MAPK/ERK kinase (MEK) inhibitor or any of the ophthalmic compositions disclosed herein.

The disclosure also provides for use of an effective amount of a MAPK/ERK kinase (MEK) inhibitor or any of the ophthalmic compositions disclosed herein for the preparation of a medicament for the reducing or preventing scarring, opacification, or neovascularization in the cornea or the surface of the eye of a subject.

The disclosure provides for a composition for reducing or preventing scarring, opacification, or neovascularization in the cornea or the surface of the eye of a subject, wherein the composition comprises an effective amount of a MAPK/ERK kinase (MEK) inhibitor or any of the ophthalmic compositions disclosed herein.

In another embodiment, the disclosure provides for methods of improving vision or reducing limitations on vision in a subject in need, wherein the method comprises administering to the subject an effective amount of a MAPK/ERK kinase (MEK) inhibitor or any of the ophthalmic compositions disclosed herein.

The disclosure also provides for use of an effective amount of a MAPK/ERK kinase (MEK) inhibitor or any of the ophthalmic compositions disclosed herein for the preparation of a medicament for improving vision or reducing limitations on vision in a subject in need.

The disclosure provides for a composition for improving vision or reducing limitations on vision in a subject in need, wherein the composition comprises an effective amount of a MAPK/ERK kinase (MEK) inhibitor or any of the ophthalmic compositions disclosed herein.

In any of methods, uses and compositions disclosed herein, the subject is suffering from aniridia, scarring on the surface of the eye such as corneal scarring, opacification on the surface of the eye, neovascularization on the surface of the eye such as corneal neovascularization, ocular surface inflammation, severe ocular surface injuries, limbal stem cell deficiency, cancers, infections, auto-immune disease, inflammatory diseases or congenital diseases associated with reduced expression of PAX6.

A method of treating an ocular disease or condition in a subject in need, wherein the method comprises administering to the subject an effective amount of a MAPK/ERK kinase (MEK) inhibitor.

The disclosure also provides for use of an effective amount of a MAPK/ERK kinase (MEK) inhibitor or any of the ophthalmic compositions disclosed herein for the preparation of a medicament for treating an ocular disease or condition in a subject in need.

In addition, the disclosure provides for a composition for treating an ocular disease or condition in a subject in need, wherein the composition comprises an effective amount of a MAPK/ERK kinase (MEK) inhibitor or any of the ophthalmic compositions disclosed herein.

In any of the methods, uses or composition the ocular disease or condition treated or prevented is aniridia, scarring on the surface of the eye such as corneal scarring, opacification of the surface of the eye, neovascularization on the surface of the eye such as corneal neovascularization, ocular surface inflammation, severe ocular surface injuries, limbal stem cell deficiency, cancers, infections, auto-immune disease, inflammatory diseases or congenital diseases associated with reduced expression of PAX6.

In any of the methods, uses and compositions disclosed herein, the MEK inhibitor is a MEK1 or MEK2 inhibitor is PD098059, PD0325901, Trametinib, Cobimetinib, MEK 1/2 inhibitor AS703988, MEK inhibitor AZD8330, MEK inhibitor CS3006, MEK inhibitor RO4987655, MEK inhibitor SHR 7390, MEK inhibitor TAK-733, MEK/Aurora kinase dual inhibitor BI 847325, MEK-1/MEKK-1 inhibitor E6201, refametinib, selumetinib, binimetinib, CI-1040, AZD6244, GDC-0973, GSK1120212, AS703026, BS203580, MEK162, GDC-0623, RO5126766, WX-554, HL-085, or U0126.

In any of the methods, uses and compositions disclosed herein, the MEK inhibitor is an ERK inhibitor, such as is FRI-20, ON-01060, VTX-11e, 25-OH-D3-3-BE, B3CD, bromoacetoxycalcidiol, 180204 AEZ-131, AEZS-131, AEZS-136, SCH-772984, AZ-13767370, BL-EI-001, LY-3214996, LTT-462, KO-947 CC-90003, GDC-0994, RG-7842, MK-8353, SCH900353, BVD-523, or Ulixertinib.

In any of the methods, medicament and compositions disclosed herein, the MAPK/ERK kinase (MEK) inhibitor is administered by topical route or oral route.

In any of the disclosed methods, uses or medicaments, the MEK inhibitor is in a composition comprising about 0.1 to about 10 μM MEK inhibitor, about 1 to about 20% dimethyl sulfoxide (DMSO) and about 1 to about 20% hydroxypropyl methylcellulose (HPMC). In another embodiment, the ophthalmic composition comprises about 0.1 to about 10 μM MEK inhibitor, about 10% to about 50% poloxamer, such as poloxamer 407 (also known as pluronic F127). In particular embodiments, the MEK inhibitor is PD0325901. An exemplary composition is about 1 μm PD0325901, about 2% DMSO, about 20% HPMC, in PBS. Another exemplary composition is about 1 μm PD0325901, about 30% poloxamer 407, in PBS. In addition, this exemplary composition further comprises an ophthalmic excipient, such as polyvinyl alcohol, povidone, hydroxypropyl methyl cellulose, poloxamers, polyols, carbopol, pluronics, carbomers, carboxymethyl cellulose, hydroxyethyl cellulose, cyclodextrins, phosphate buffer, citrate buffer, tris buffer, sodium chloride, potassium chloride, polysorbate 80, vegetable oil, preservative or a combination thereof.

In any of the methods, uses and compositions, the MEK inhibitor is administered at a concentration ranging from about 0.1 to about 10 μM MEK inhibitor, or about 0.2 to about 20 μM MEK inhibitor, or about 0.05 to about 50 μM MEK inhibitor, or 0.1 to about 5 μM MEK inhibitor, or 0.1 to about 1 μM MEK inhibitor, or about 0.25 to about 8 μM MEK inhibitor, or about 0.25 to about 6 μM MEK inhibitor, or 0.25 to about 5 μM MEK inhibitor, or about 0.25 to about 3 μM MEK inhibitor, or about 0.25 to about 1 μM MEK inhibitor, or about 0.5 to about 20 μM MEK inhibitor, or about 0.5 to about 10 μM MEK inhibitor, or about 0.5 to about 8 μM MEK inhibitor, or 0.5 to about 5 μM MEK inhibitor, or 0.5 to about 3 μM MEK inhibitor, or 0.5 to about 1 μM MEK inhibitor, or about 1 to about 5 μM MEK inhibitor, or about 1 to about 10 μM MEK inhibitor, or about 1 to about 20 μM MEK inhibitor, or 1 to about 50 μM MEK inhibitor, or 5 to about 10 μM MEK inhibitor, or 5 to about 20 μM MEK inhibitor. Exemplary concentrations of MEK inhibitor in the disclosed compositions include about 0.1 μM MEK inhibitor, about 0.2 μM MEK inhibitor, about 0.2 μM MEK inhibitor, about 0.4 μM MEK inhibitor, about 0.5 μM MEK inhibitor, about 0.6 μM MEK inhibitor, about 0.7 μM MEK inhibitor, or about 0.8 μM MEK inhibitor, or about 0.9 μM MEK inhibitor, about 1 μM MEK inhibitor, about 2 μM MEK inhibitor, about 3 μM MEK inhibitor, about 4 μM MEK inhibitor, about 5 μM MEK inhibitor, about 6 μM MEK inhibitor, about 7 μM MEK inhibitor, about 8 μM MEK inhibitor, about 9 μM MEK inhibitor, about 10 μM MEK inhibitor, about 12 μM MEK inhibitor, about 15 μM MEK inhibitor, about 18 μM MEK inhibitor, about 20 μM MEK inhibitor, about 30 μM MEK inhibitor, about 40 μM MEK inhibitor or about 50 μM MEK inhibitor.

In any of the disclosed methods, uses and compositions, the MEK inhibitor is in a composition further comprising about 1% to about 20% DMSO, or about 1% to about 20% DMSO, 2% to about 30% DMSO, or about 3% to about 40% DMSO, or about 4% to about 50% DMSO, or about 1% to about 10% DMSO, or about 1% to about 5% DMSO, or about 2% to about 20% DMSO, or about 2% to about 10% DMSO, or about 2% to about 5% DMSO, or about 5% to about 10% DMSO, or about 5% to about 20% DMSO, or about 5% to about 50% DMSO, or about 10% to about 20% DMSO, or about 12% to about 20% DMSO, or about 15% to about 20% DMSO, or about 20% to about 50% DMSO. Exemplary DMSO concentration include 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40% or 50%.

In any of the disclosed methods, uses and compositions, the MEK inhibitor is in a composition further comprising about 1% to about 20% HPMC, or about 1% to about 20% HPMC, 2% to about 30% HPMC, or about 3% to about 40% HPMC, or about 4% to about 50% HPMC, or about 1% to about 10% HPMC, or about 1% to about 5% HPMC, or about 2% to about 20% HPMC, or about 2% to about 10% HPMC, or about 2% to about 5% HPMC, or about 5% to about 10% HPMC, or about 5% to about 20% HPMC, or about 5% to about 50% HPMC, or about 10% to about 20% HPMC, or about 12% to about 20% HPMC, or about 15% to about 20% HPMC, or about 20% to about 50% HPMC. Exemplary HPMC concentration include 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40% or 50%.

In addition, the disclosed methods, uses and composition, the MEK inhibitor is in a composition further comprising a poloxamer as a solvent, such as poloxamer 407 (also known as pluronic F127). For example, the disclosed comprises about 1% to about 20% poloxamer, or about 1% to about 20% poloxamer, 2% to about 30% poloxamer, or about 3% to about 40% poloxamer, or about 4% to about 50% poloxamer, or about 1% to about 10% poloxamer, or about 1% to about 5% poloxamer, or about 2% to about 20% poloxamer, or about 2% to about 10% poloxamer, or about 2% to about 5% poloxamer, or about 5% to about 10% poloxamer, or about 5% to about 20% poloxamer, or about 5% to about 50% poloxamer, or about 10% to about 20% poloxamer, or about 12% to about 20% poloxamer, or about 15% to about 20% poloxamer, or about 20% to about 50% poloxamer. Exemplary poloxamer concentrations include 1%, 2%, 3%, 4%, 5%, 6%, 7%, 8%, 9%, 10%, 11%, 12%, 13%, 14%, 15%, 16%, 17%, 18%, 19%, 20%, 25%, 30%, 40% or 50%.

### BRIEF DESCRIPTION OF THE DRAWING

FIG. 1 demonstrates the effect of MEK inhibition on PAX6 expression. (A) PAX6 mRNA measurement and (B) PAX6 protein measurement in human corneal epithelial cells treated with PD0325901, showing an increase in PAX6 expression following MEK inhibitor treatment (n=3). (C) PAX6 mRNA measurement in murine PAX6 heterozygote corneal MSCs treated with PD0325901, showing an increase in PAX6 expression following MEK inhibitor treatment (n=3). (D) Confirming specific ERK1/ERK2 inhibition by PD0325901 in human corneal epithelial cells (n=3). (E) PAX6 protein measurement in human corneal epithelial cells treated with two other MEK inhibitor, Cobimetinib and Refametinib, showing other MEK inhibitors can induce PAX6 expression as well (n=3). Significance determined by t test. *P<0.05. Ctrl: control. RX: treatment.

FIG. 2 provides postnatal 30 evaluation of Pax6^{sey-Neu/+} mice treated with MEK inhibitor. (A) Color photo, (B) optical coherence tomography (OCT), (C, D) H&E staining, (E) Epithelial thickness, (F) stromal thickness and (G) scarring ratio of mice eyes treated with systemic or topical PD0325901 (n=6). Note the difference in the thickness of the corneal layers, as well as the severity of adhesions. The thickness measurements were done in the central cornea immediately outside the scar area (the lines in the color photos do not necessary indicate with the area of measurement). Scale bar: 50 μm. Significance determined by t test. *P<0.05. Ctrl: vehicle control. RX: treatment. Arrow: central cornea. Asterisk: lens. Arrowhead: iris. e: epithelium. s: stroma.

FIG. 3 provides immunostaining of Pax6^{sey-Neu/+} mice corneas to assess the effect of MEK inhibitor. (A) Comparing topical MEK inhibitor with topical vehicle control on P30 showing increased PAX6 staining in the corneal epithelium, as well as (B) more abundant cytokeratin (CK)12 representing favorable differentiation. Wild type cornea immunostaining used as a reference. (C, E) CK12 immunostaining of corneal whole mount at P90 showing a more normal differentiation pattern in the topically (MEK inhibitor) treated corneas. Blue: DAPI. Red: CK12. (D, F) Ki67 immunostaining of mice corneas on P30 showing significant decrease in abnormal proliferation of corneal epithelium following topical MEK inhibitor treatment. Blue: DAPI. Green: Ki67. *P<0.05. ***P<0.001. RX: MEK inhibitor treatment. Veh: vehicle control.

FIG. 4 provides electoretinography in response to light stimulation. (A) Comparing topical MEK inhibitor with topical vehicle control on P30 showing increased PAX6 staining in the corneal epithelium, as well as (B) more abundant cytokeratin (CK)12 representing favorable differentiation. Wild type cornea immunostaining used as a reference. (C, E) CK12 immunostaining of corneal whole mount at P90 showing a more normal differentiation pattern in the topically (MEK inhibitor) treated corneas. Blue: DAPI. Red: CK12. (D, F) Ki67 immunostaining of mice corneas on P30 showing significant decrease in abnormal proliferation of corneal epithelium following topical MEK inhibitor treatment. Blue: DAPI. Green: Ki67. *P<0.05. ***P<0.001. RX: MEK inhibitor treatment. Veh: vehicle control.

FIG. 5 demonstrates the effect of MEK inhibition on limbal stem cell deficiency. Treatment with PD0325901 led to significantly less corneal haziness and neovascularization up to 1 month after injury.

### DETAILED DESCRIPTION

MAPK/ERK kinase (MEK) inhibitors are anti-proliferative medications widely used in the clinic to limit progression of several neoplastic diseases, and they have been shown to reduce VEGF pathway stimulation. The disclosure provides evidence showing that MEK inhibitors can limit the corneal scarring, opacification and neovascularization in severe corneal injury and disease. In accordance with the principles herein MEK inhibitors can help reduce scarring and neovascularization in an animal model in part by stimulating PAX6 expression. Pax6 is one of the major regulators of eye development and is important in tissue regeneration. As an example, a treatment formula using MEK inhibitors has successfully prevented progression of several corneal manifestations in a mouse model of aniridia (a human condition with PAX6 mutation that develops corneal scarring and neovascularization), and promoted a clear cornea and improved ocular function.

Many ocular diseases and insults can lead to corneal opacification, scarring and neovascularization. Some examples of the diseases and conditions include ocular surface inflammation, severe ocular surface injuries, limbal stem cell deficiency, cancers, infections or immune diseases that affect the cornea or the surface of the eye, inflammatory diseases that affect the cornea or the surface of the eye, as well as congenital diseases such as aniridia. MEK inhibitors can be employed, in accordance with the principles herein, to prevent the mentioned complications from developing, minimize their further progression, or at the very least reduce the intensity of the problems, in order to preserve vision as much as possible. This approach can limit the aforementioned complications and improve ocular function in an in vivo murine model.

Reducing corneal scarring and neovascularization will lead to better vision and reduce the limitations caused by the related complications. For instance, although aniridia is a congenital problem, it is shown that the corneal disease continues to develop and worsen after birth. The studies conducted in accordance with the principles herein have shown that MEK inhibition can prevent further progression of these complications, via significantly reducing corneal scarring and neovascularization, as well as epithelial differentiation improvement. Many ocular surface injuries can lead to corneal scarring and neovascularization, as can conditions such as limbal stem cell deficiency. MEK inhibition can reduce corneal opacification and neovascularization following severe epithelial and limbal injuries, opening a new approach toward limiting ocular surface injuries and limbal stem cell deficiency complications.

Thus, the use of a topical medication for preventing corneal scarring and neovascularization is set forth. Some examples of the diseases and conditions include ocular surface inflammation, severe ocular surface injuries, limbal stem cell deficiency, as well as congenital diseases. Corneal scarring is partially due to corneal fibroblasts' reproduction and maldifferentiation. Neovascularization is partially due to VEGF pathway stimulation in inflammatory conditions following injury. MAKK/ERK kinase (MEK) inhibitors are anti-proliferative medications used in the clinic to limit progression of several neoplastic diseases, and they have been shown to reduce VEGF pathway stimulation.

In accordance with the principles herein, MEK inhibitors, specifically the optimized topical formulation herein, can limit scarring, opacification and neovascularization in severe corneal injury and disease. In accordance with the principles herein, MEK inhibitors can help reduce scarring and neovascularization in an animal model in part by stimulating PAX6 expression. PAX6 is one of the major regulators of eye development and is important in tissue regeneration.

Several MEK inhibitors were tested in vitro, in human corneal epithelial cells, and found that they all can induce PAX6 production to a desirable extent. MEK inhibitors were also further tested in vivo, in a mouse model of aniridia (a human condition with PAX6 mutation that develops corneal scarring and neovascularization), in order to optimize a topical treatment formulation. Using MEK inhibitors as a topical medication has not been reported before. A topical treatment was optimized using a suitable MEK inhibitor, such as pd0325901, a potent MEK inhibitor. A novel dosage and formulation of the drug, its safety were studied in our animal model. Our exemplary optimized formulation and dosage resulted in no toxicity, with an exemplary formulation up to 1 μm PD, 2% DMSO, 2% HPMC, in PBS. Compositions containing a variety of carriers and an MEK inhibitor with our without the following constituents are proposed: for example, compositions can include constituents in the range of 0.1 to 10 μm PD, 1 to 20% DMSO, 1 to 20% HPMC, in PBS are contemplated in accordance with the principles herein.

Using the exemplary topical formulation above, progression of the severe corneal manifestations in a mouse model of aniridia was successfully prevented and promoted a clear cornea and improved ocular function).

The formulation was tested in an epithelial and limbal stem cell injury model, in which we injured the epithelium of the cornea up to the limbal area, creating a severe scar forming wound with limbal stem cell deficiency. The formulation successfully reduced the corneal opacity and neovascularization in this model as well.

One of the models that we used to study the corneal opacification, scarring and neovascularization, was aniridia mouse model; a human condition with PAX6 mutation that develops corneal scarring and neovascularization.

MEK pathway inhibits the PAX6 pathway, therefore MEK inhibitors were used to indirectly stimulate PAX6 production. Using PAX6 induction as a treatment for aniridia, corneal scarring and neovascularization has not been reported before. MEK inhibition increased PAX6 mRNA and protein expressions in human corneal epithelial cells (2.7±0.2 & 3.0±0.3 folds).

### MEK/ERK Kinase Inhibitors

The term “MAPK/ERK kinase inhibitor” refers to an agent that inhibits activity or expression of the MEK1 and/or MEK2 kinase or inhibits the activity of ERK1 or ERK2 kinase. In particular, a MAPK/ERK kinase (MEK) inhibitor refer to an agent that inhibits the activity of mitogen-activated protein kinases enzymes MEK1 and/or MEK2. MEK1 and MEK2. The Ras-dependent Raf/MEK/ERK1/2 mitogen-activated protein (MAP) kinase signaling pathway is known to be a major regulator of cell proliferation and cell survival. MEK is a MAPKK that activates a MAPK (ERK), which is the final kinase in the RAS-RAF-MEK-ERK signaling pathway. In this signaling pathway, the tyrosine kinase Raf acts as a MAP kinase kinase kinase (MAPKKK) and activates the MAP kinase kinases (MAPKKs) MEK1 and MEK2, which, in turn, catalyze the activation of the effector MAP kinases ERK1 and ERK2. Once activated, ERK1/ERK2 phosphorylate a panoply of nuclear and cytoplasmic substrates involved in diverse cellular responses, such as cell proliferation, survival, differentiation, motility, and angiogenesis.

MEK1 and MEK2 amino acids are 86% identical, which is why many of the MEK1/2 inhibitors developed are not selective for either isoform. MEK1 and MEK2 belong to the family of MAPKKs (also known as MEKs or MKKs), which are dual specificity enzymes that phosphorylate threonine and tyrosine residues within the activation loop of their MAP kinase substrates.

Exemplary MEK1/MEK2 include inhibitors include PD098059, PD0325901 (Mirdametinib), Trametinib (MEKkinist), Cobimetinib (also known as GDC-0973; XL518 or Cotellic), MEK 1/2 inhibitor AS703988 (also known as AS703988/MSC2015103B, MSC2015103B), MEK inhibitor AZD8330 (ARRY-424704), MEK inhibitor CS3006 (MAPK kinase inhibitor CS3006), MEK inhibitor RO4987655, MEK inhibitor SHR 7390 (also known as MEKi SHR 7390), MEK inhibitor TAK-733, MEK/Aurora kinase dual inhibitor BI 847325, MEK-1/MEKK-1 inhibitor E6201, pimasertib (MEK inhibitor AS703026, MSC1936369B/AS703026), refametinib (MEK inhibitor RDEA119, BAY 869766), selumetinib (ARRY-142886, AZD6244 or Koselugo), CI-1040, AZD6244, GDC-0973, GSK1120212, AZD8330, RO4987655, AS703026, BS203580, MEK162 (Binimetinib, Mektovi, ARRY-162, ARRY-438162), GDC-0623, RO5126766, WX-554, HL-085, or U0126.

PD09805059 is a small molecule inhibitor of MEK1/MEK2 PD98059 binds to the inactive form of MAPKK and prevents activation by upstream activators such as c-Raf. In particular, this agent inhibits the dephosphorylated form of MEK 1 and MEK1 mutant S217E and S221E). Cobimetinib (GDC-0973; XL518 or Cotellic) is a small molecule oral inhibitor of MEK1 and MEK2. Cobimetinib specifically binds to and inhibits the catalytic activity of MEK1, resulting in inhibition of extracellular signal-related kinase 2 (ERK2) phosphorylation and activation and decreased tumor cell proliferation. Rafametinib (RDEA119/BAY 869766) is an allosteric MEK inhibitor. Rafametinib selectively binds directly to an allosteric pocket in the MEK1/2 enzymes. This agent is highly efficacious at inhibiting cell proliferation in several tumor cell lines in vitro. In vivo, RDEA119/BAY 869766 exhibits potent activity in xenograft models of melanoma, colon, and epidermal carcinoma. Rafametinib exhibits complete suppression of ERK phosphorylation at fully efficacious doses in mice.

Exemplary ERK inhibitors include FRI-20, ON-01060, VTX-11E, 25-OH-D3-3-BE, B3CD, bromoacetoxycalcidiol, 180204 AEZ-131, AEZS-131, AEZS-136, SCH-772984, AZ-13767370, BL-EI-001, LY-3214996, LTT-462, KO-947 CC-90003, GDC-0994, RG-7842, MK-8353, SCH900353, BVD-523, and ulixertinib.

### Ophthalmic Compositions

Provided herein is an exemplary optimized composition of up to 1 μM of MEK/ERK inhibitor of MEK/ERK inhibitor, 2% DMSO, 2% HPMC, in PBS and the dosage of MEK/ERK inhibitor resulted in no toxicity. Compositions containing a variety of carriers and an MEK inhibitor with our without the following constituents are proposed: for example, compositions can include constituents in the range of 0.1 to 10 uM PD, 1 to 20% DMSO, 1 to 20% HPMC, in PBS are contemplated in accordance with the principles herein.

“Pharmaceutically acceptable excipient” or “pharmaceutically acceptable ophthalmic excipient” means an excipient that is useful in preparing a pharmaceutical composition of the disclosure. Such an excipient is considered by one skilled in the art as being generally safe, non-toxic and neither biologically active nor otherwise undesirable, and includes excipient that is acceptable for veterinary use as well as human pharmaceutical use. A “pharmaceutically acceptable excipient” as used in the specification and claims includes both one and more than one such excipients. Exemplary pharmaceutically acceptable excipients include a salt (such as sodium chloride) or a tonicity agent, gum, resin, a solvent such as water, a non-aqueous solvents (such as an alcohol, an oil, a buffer solution to maintain pH, a pH modifying agent (e.g., a base such as sodium hydroxide, and an acid such as hydrochloric acid), an emulsifier, a thickening agent, a micro or a nano-emulsion forming agent, a preservative, a surfactant, etc. Exemplary pharmaceutically acceptable excipients that can be used in ophthalmic compositions of the disclosure include, but are not limited to, water, benzyl alcohol, sodium hydroxide, hydrochloric acid, Castrol oil, citrate buffer, Tris buffer, phosphate buffer, as well as other excipients known to one skilled in the art.

In some embodiments, ophthalmic compositions of the disclosure can also include salts such as sodium chloride. Yet in other embodiments, the ophthalmic compositions of the disclosure can also include a non-aqueous solvent such as benzyl alcohol, ethanol, or other non-aqueous solvents known to one skilled in the art.

Still in other embodiments, pH of the ophthalmic compositions of the disclosure is adjusted from pH of about 5.0 to pH of about 8.5, typically from pH of about 5.0 to pH of about 8.0, and often from pH of about 5.0 to pH of about 7.5. Additional, exemplary pH rangers are about pH 6 to about pH 8 or about pH 6.2 to about pH 7.2, about pH 6.4 to about pH 7.4, or about pH 6.5 to about pH 7.5, about pH 6.6 to about pH 7.6, about pH 6.8 to about pH 7.8. For example, the pH is about 5.0. is about 5.1, or about 5.2, or about 5.3, or about 5.4, or about 5.5, or about 5.6, or about 5.7, or about 5.8, or about 5.9 about 6.0, or about 6.1, or about 6.2, or about 6.3, or about 6.4, or about 6.5, or about 6.6, or about 6.7, or about 6.8, or about 6.9, or about 7.0, or about 7.1, or about 7.2, or about 7.3, or about 7.4, or about 7.5, or about 7.6, or about 7.7, or about 7.8, or about 7.9, or about 8.0. The pH of ophthalmic compositions of the disclosure can be adjusted using, for example, sodium hydroxide and/or hydrochloric acid as needed to achieve a desired pH level.

The ophthalmic compositions can be formulated as an eye drop, topical liquid, an ointment, emulsion, suspension or a gel (e.g., IgG sodium in hydrogel). The ophthalmic compositions can also be formulated as a nano-emulsion of oil or a suspension. In addition, the ophthalmic compositions can be formulated as an injectable compositions.

In some embodiments, ophthalmic compositions of the disclosure are preservative free and are formulated for a single-use or in a multi-dose vials. If a preservative is used, suitable preservatives include, but are not limited to, benzalkonium, purite, chlorobutanol, sodium perborate, stabilized oxychloro complex (SOC), Polyquaternium-1 (Polyquad, PQ-1), Thimerosal, Benzyl alcohol, Sorbic acid, Methyl/propyl paraben, Chlorhexidine, Disodium EDTA, sofZia, and other preservatives known to one skilled in the art of ophthalmology or ophthalmic composition chemistry.

Ophthalmic compositions of the disclosure can be homogeneous or heterogeneous. In some embodiments, ophthalmic compositions of the disclosure contain an oil or a fatty acid ester. A fatty acid ester has the meaning commonly understood in the art, being an ester formed between an alcohol and a fatty acid. Exemplary fatty acid esters that are useful in compositions of the disclosure include, but are not limited to, triglyceride esters commonly known as vegetable oils, mono and diglyceride esters of fatty acids, fatty acid methyl esters, as well as other fatty acid esters that are known to one skilled in the art. It should be appreciated the fatty acid ester can be a mixture of several chemical compounds or an essentially pure compound. Typically, the fatty acid ester is a vegetable oil. Particular examples of vegetable oils that can be used include, but are not limited to, castor oil, sesame oil, soybean oil, cottonseed oil, olive oil, peanut oil, safflower oil, sunflower oil, palm oil, palm kernel oil, canola oil, and Miglyol Oil®.

Various vehicles can be used in the ophthalmic compositions of the disclosure. These vehicles include, but are not limited to, purified water (water), polyvinyl alcohol, povidone, hydroxypropyl methyl cellulose, poloxamers, carboxymethyl cellulose, hydroxyethyl cellulose, polyols, sodium hyaluronate, pluronics, corbopol, cyclodextrin and a mixture of two or more thereof. The vehicle is used in the compositions in amounts as needed to provide the concentration of the active compound(s) disclosed herein. In one particular embodiment, the vehicle comprises water.

In some embodiments of this disclosure, an emulsion stabilizing polymer is used. While not intending to limit the scope of the disclosure, emulsion stabilizing polymers generally contain hydrophilic groups such as cellulose, sugars, ethylene oxide, hydroxide, carboxylic acids or other polyelectrolytes. Without being bound by any theory, it is believed that these polymers help to stabilize emulsions by increasing the viscosity of the composition as well as by reducing the interfacial tension. Surfactants such as polysorbate 80 or other surfactants acceptable for Opthalmics can be used to stabilize emulsions, Some examples of emulsion stabilizing polymers useful in this disclosure include, but are not limited to, carbomers, Pemulen®, sodium carboxymethylcellulose, hydroxypropylmethylcellulose, povidone, polyvinyl alcohol, polyethylene glycol and a mixture of two or more thereof.

The ophthalmic composition of the present disclosure can be packaged in various package forms known in the field of topical ophthalmic. In one particular embodiment, the ophthalmic composition is packaged in sterile, preservative-free single-use packs or vials or containers (i.e., the unit dose vials). Each vial, for example as small as a 0.9 mL, may be made of low density polyethylene so as to contain a small quantity of the composition, e.g., 0.4 mL for a single use. This way, where the ophthalmic composition is sterilized and contained in disposable single-dose containers for topical use in drop form, multiple vials in the form of a set of 30 vials, 60 vials and so on can be packaged in a tray with a lid, for example, a polypropylene tray with an aluminum peelable lid. The entire contents of each tray can be dispensed intact, and one vial or pack is used each time and immediately discarded after each use. For example, plastic ampules or vials or containers can be manufactured using blow-fill-seal (BFS) technology. The BFS processes may involve plastic extrusion, molding, aseptic filling, and hermetic sealing in one sequential operation and those processes are known in the art. In another embodiment, the composition is packaged in multi-dose vials such that the materials can be dispensed as sterile at each time using specialized container/closure maintaining the sterility integrity. In yet another embodiment, the ophthalmic composition is packaged in conventional vials/containers as a sterile product.

In some embodiments, the dosage form of the disclosure is eye drops of heterogeneous aqueous solution, eye drop compositions.

### Pharmaceutical Formulations and Modes of Administration

As used herein, “subject” refers to any mammal, including humans, domestic and farm animals, and zoo, sports, or pet animals, such as dogs, horses, cats, sheep, pigs, cows, etc. The preferred mammal herein is a human, including adults, children, and the elderly. Preferred sports animals are horses and dogs. Preferred farm animals are cows, pigs, horses, goats and sheep. Preferred pet animals are dogs and cats.

As used herein, an “effective amount” or a “therapeutically effective amount” in reference to the disclosed compositions refers to the amount of MAPK/ERK kinase (MEK) inhibitor sufficient to induce a desired biological, pharmaceutical, or therapeutic result. That result can be treating, reducing or preventing of the signs, symptoms, or causes of a disease or disorder or condition, or any other desired alteration of a biological system. For example, a therapeutically effective amount of a MAPK/ERK kinase (MEK) inhibitor reduces or prevents scarring, neovascularization, or opacification. In addition, a therapeutically effective amount of MAPK/ERK kinase (MEK) reduces cell proliferation. In some embodiments, a therapeutically effective amount of MAPK/ERK kinase inhibitor (MEK) improves vision and/or reduces, prevents or treats the signs, symptoms or causes of an ocular disease or condition. In particular, a therapeutically effective amount of a MAPK/ERK kinase (MEK) inhibitor induces or increases expression of PAX6.

As used herein, the terms “treating” and “treatment” refer to both therapeutic treatment and prophylactic or preventative measures.

In exemplary aspects, the compositions disclosed herein is part of a pharmaceutical composition comprising a MAPK/ERK kinase (MEK) and a pharmaceutically acceptable carrier, diluent, or excipient. In exemplary aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopedia for use in animals, including humans.

The pharmaceutical composition in various aspects comprises any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In exemplary aspects, the pharmaceutical composition comprises formulation materials that are nontoxic to recipients at the dosages and concentrations employed. In specific embodiments, pharmaceutical compositions comprising MAPK/ERK kinase (MEK) inhibitor and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition comprises formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbatc, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18″ Edition, (A. R. Genrmo, ed.), 1990, Mack Publishing Company.

The pharmaceutical compositions in various instances are formulated to achieve a physiologically compatible pH. In exemplary embodiments, the pH of the pharmaceutical composition is for example between about 4 or about 5 and about 8.0 or about 4.5 and about 7.5 or about 5.0 to about 7.5. In exemplary embodiments, the pH of the pharmaceutical composition is between 5.5 and 7.5.

The pharmaceutical composition may be administered to a subject via parenteral, nasal, oral, pulmonary, topical, vaginal, or rectal administration. The following discussion on routes of administration is merely provided to illustrate exemplary embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. A MAPK/ERK kinase (MEK) inhibitor in various instances is administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-I53-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight MAPK/ERK kinase (MEK) inhibitor in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)). In particular, the disclosure provides for formulations suitable for injections directly into the eye including subconjunctival, intraocular injection, and intraretinal injection.

Formulations suitable for oral administration in some aspects comprise (a) liquid solutions, such as an effective amount of MAPK/ERK kinase (MEK) inhibitor dissolved in diluents, such as water, saline, or orange juice; (b) capsules, sachets, tablets, lozenges, and troches, each containing a predetermined amount of HPAC, as solids or granules; (c) powders; (d) suspensions in an appropriate liquid; and (e) suitable emulsions. Liquid formulations in some aspects include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise MAPK/ERK kinase (MEK) inhibitor in a flavor, usually sucrose and acacia or tragacanth, as well as pastilles comprising MAPK/ERK kinase (MEK) inhibitor in an inert base, such as gelatin and glycerin, or sucrose and acacia, emulsions, gels, and the like containing, in addition to, such excipients as are known in the art.

### Topical Delivery

In some embodiments, a composition is formulated for administration by a topical route, for example, as a topical (e.g., dermal) formulation. In some embodiments, a composition is formulated, for example, for topical administration to a mammal. A topical formulation may include, for example, a formulation such as a gel formulation, a cream formulation, emulsions, suspensions, a lotion formulation, a paste formulation, an ointment formulation, an oil formulation, and a foam formulation. The modes of topical administration include topical liquid/solution eye drops, ointments or creams, using drug-loaded contact lenses, intraocular implants, microneedles and aerosol mists. The composition further may include, for example, an absorption emollient or a penetration enhancer.

Additional examples of a composition can optionally be formulated to be delivered to the mucosum, or by inhalation, respiration, intranasal, oral, buccal, or sublingual. Salts may be added. Non-limiting examples of salts include acetate, benzoate, besylate, bitartate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulphate, mucate, napsylate, nitrate, pamoate (embonate, phosphate, diphosphate, salicylate and disalicylate, stearate, succinate, sulphate, tartrate, tosylate, triethiodide, valerate, aluminium, benzathine, calcium, ethylene diamine, lysine, magnesium, megluminie, potassium, procaine, sodium, tromethyamine or zinc.

Topical formulations can include, for example, a liquid or cream with or without moisturizer. Components of a liquid or cream with moisturizer (moisturizing formulation) can be: Colloidal oatmeal, niacinamide, ceramides, phospholipids, triglycerides, fats or fatty acids, free fatty alcohols, waxes (esters, diesters, triesters, etc.), hydroxyacid diesters, squalene, sterol esters, cholesterol, lactones, etc. In addition, the topical formulations can be incorporated as creams, gels, or foams to serve as topical treatment for viral infection or for rectal or vaginal application (e.g. to mucosal surfaces).

Suitable carriers include: pluronic gels, polaxamer gels, hydrogels containing cellulose derivatives, including hydroxyethyl cellulose, hydroxymethyl cellulose, carboxymethyl cellulose, hydroxypropylmethyl cellulose and mixtures thereof, and hydrogels containing polyacrylic acid (Carbopols). Suitable carriers also include creams/ointments used for topical pharmaceutical preparations, e.g., creams based on cetomacrogol emulsifying ointment. The above carriers may include or exclude, for example, alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

### Ocular Diseases and Conditions

The disclosure provides for method of treating and preventing scarring on the surface on the eye such as corneal scarring, opacification, neovascularization on the surface of the eye such as corneal neovascularization, ocular surface inflammation, severe ocular surface injuries, infections, immune diseases that lead to scarring on cornea or the surface of the eye, limbal stem cell deficiency, congenital diseases associated with reduced expression of PAX6.

Defects in PAX6 gene can affect eye development and result in a broad range of clinical phenotypes. The disclosure provides for methods of treating, reducing and preventing the ocular phenotypes associated with reduced PAX6 expression such as Aniridia, Peter's anomaly, Coloboma, microphthalmia (small eyes), WAGR syndrome, optic nerve anomalies such as an underdeveloped optic nerves, anterior segment dysgenesis, ectopia papillae, hypoplasia of the iris, nystagmus (involuntary eye movements), foveal hypoplasia (underdevelopment of the region at the back of the eye responsible for sharp vision), cataracts (clouding of the lens of the eye), inflammation of the front surface of the eye (keratitis), glaucoma and corneal keratopathy. Coloboma is a gap or split in structures that make up the eye.

Aniridia, a pan ocular disorder that is primarily characterized by the absence or hypoplasia of the iris, nystagmus, and foveal hypoplasia, accompanied by cataracts, glaucoma and corneal keratopathy. Aniridia is a congenital panocular condition caused by a mutation in one copy of the PAX6 gene. Although the ocular problems begin in utero and are present at birth, early postnatal stimulation of the normal copy of PAX6 may prevent or reduce further progression of the disease.

Reduced expression of PAX6 also results in Peter's anomaly, which is a condition characterized by the abnormal development of certain structures at the front of the eye and clouding of the clear front surface of the eye (cornea). The mutations that cause Peters anomaly reduce the PAX6 protein's ability to bind to DNA, disrupting its role as a transcription factor. As a result, normal development of the eye is impaired, leading to the features of Peters anomaly.

The PAX6 gene is located in a region of chromosome 11 that is deleted in people with WAGR syndrome, which is a disorder that affects many body systems and is named for its main features: a childhood kidney cancer known as Wilms tumor, an eye problem called anirida, genitourinary anomalies, and intellectual disability (formerly referred to as mental retardation). As a result of this deletion, affected individuals are missing one copy of the PAX6 gene in each cell. A loss of the PAX6 gene is associated with the characteristic eye features of WAGR syndrome, including aniridia, and may affect brain development.

Infections and diseases that cause scarring and neovascularization on the surface of the eye include viral infections such as herpes simplex, herpes zoster, bacterial infections such as staphylococcus, pseudomonas, syphilis, fungal infections and other immune mediated keratitis. Neovascularization is also caused by inflammation related to injury and traumatic conditions. In addition, neovascularization is caused by trachoma, conreal ulcers, phylctenular keratoconjunctivitis, rosacea keratiitis, interstitial keratiitis, pterygium, chemical burns and wearing contact lenses for over-extended periods of time.

Scarring and neovascularization is also caused by cancers on the surface of the eye or conjunctival cancers. Eye cancers include squamous carcinoma (ocular surface squamous neoplasia), ocular surface melanocytic tumors, malignant melanoma, conjunctival melanoma, eyelid carcinoma, lymphoma such intraocular lymphoma and Non-Hodgkin lymphoma, and retinoblastoma.

### Sustained Release

The disclosed composition release a MAPK/ERK kinase (MEK) inhibitor by immediate release, controlled release, sustained release, extended release, delayed release, or bi-phasic release formulation. Methods of formulating compounds for controlled release are known in the art. See, for example, Qian et al., J Pharm 374: 46-52 (2009) and International Patent Application Publication Nos. WO 2008/130158, WO2004/033036; WO2000/032218; and WO 1999/040942.

As used herein, the term “sustained release” is characterized by the gradual release of the MEK inhibitor over an extended period of time, optionally greater than about 30 minutes. With sustained release, the rate of release of the MEK inhibitor is controlled in order to maintain activity of the therapeutic agents for a longer period of time. In some embodiments, greater than about 40% of the MEK inhibitor is released over a period of about 6 hours or more.

For example, sustained release compositions allow delivery of a MEK inhibitor to a subject over an extended period of time. Such release rates can provide therapeutically effective levels of the MEK inhibitor for an extended period of time and thereby provide a longer period of pharmacologic or diagnostic response as compared with conventional rapid release dosage forms. Such longer periods of response provide for many inherent benefits that are not achieved with immediate release dosages.

### EXAMPLES

### Example 1

### MEK Inhibitor Induced PAX6 Expression in Human Corneal Epithelial Cells

Several MEK inhibitors were tested in vitro, in human corneal epithelial cells, and found that they all can induce PAX6 production to a desirable extent. For example, PD0325901 (PD) was tested in human corneal epithelial cells (HCEC).

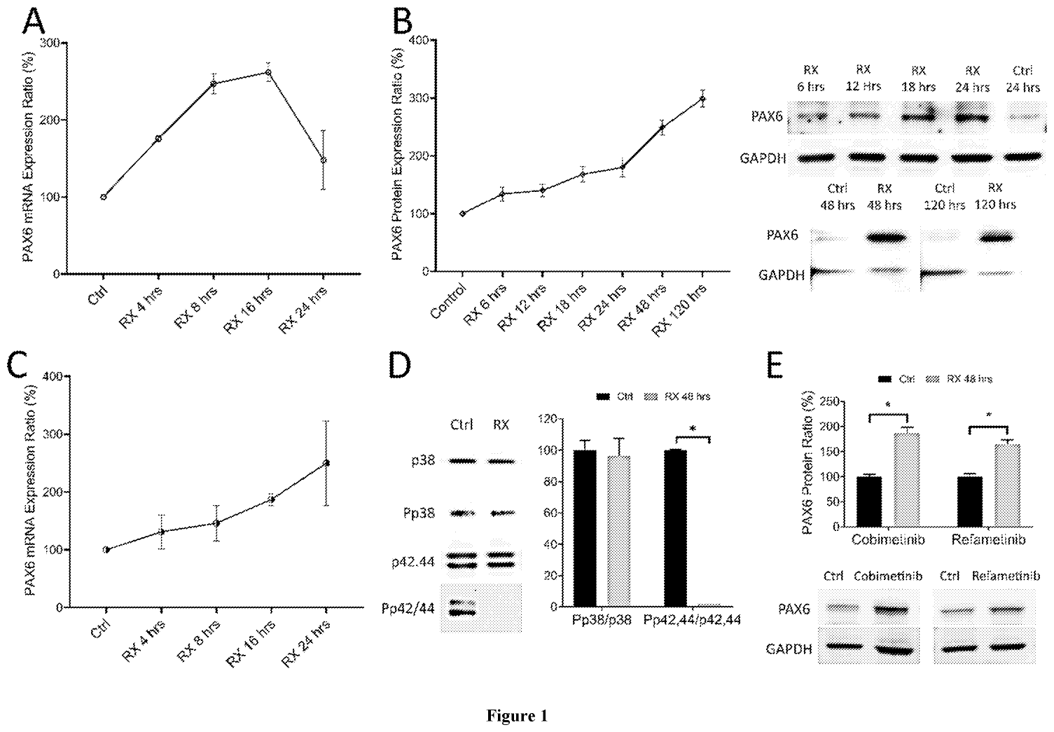

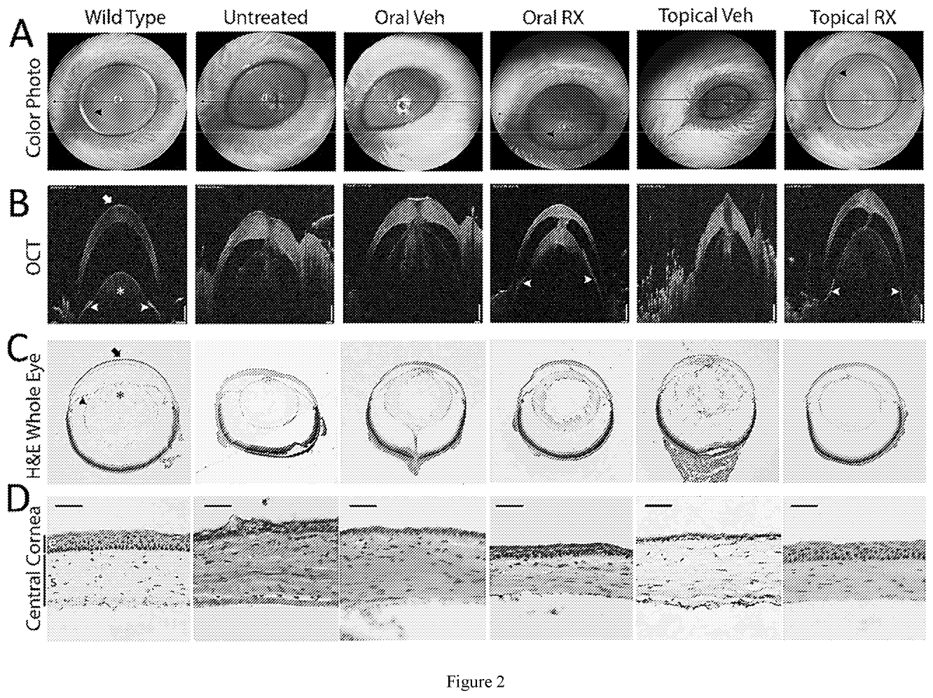

HCEC were treated with a PD composition comprising 1 μM PD dissolved in culture media for up to 120 hours. PAX6 protein levels were detected by SDS-PAGE and quantified by comparing the levels of GAPDH protein (FIG. 1A). In the same treated corneal epithelial cells, PAX6 mRNA expression was measured by RT-PCR using three different primers. (FIG. 1B).

Western blot analysis of human corneal epithelial cells confirmed that this MEK inhibitor effectively and specifically suppresses ERK1 and ERK2, while having minimal effect on p38 as the control (FIG. 10).

Additional MEK inhibitors, Cobimetinib and Rafametinib, also induced PAX6 protein expression in human cornea epithelium cells after 48 hours of treatment. As shown in FIG. 1D, the protein levels detected by SDS-PAGE were quantitated by comparing the protein level with GAPDH protein level. In summary, the MEK inhibition increased PAX6 mRNA and protein expressions in human corneal epithelial cells (2.7±0.2 & 3.0±0.3 folds).

### Example 2

### MEK Inhibitors Induced PAX6 Expression In Vivo

Aniridia is a human condition with PAX6 mutation that develops corneal scarring and neovascularization. The MEK pathway is known to inhibit the PAX6 pathway, therefore it was investigated whether MEK inhibitors can indirectly stimulate PAX6 production. MEK inhibitors were tested in vivo, in a mouse model of Aniridia which allowed for investigation corneal opacification, scarring and neovascularization. Oral and topical formulations were optimized using an exemplary MEK inhibitor, such as PD0325901 (PD). The exemplary optimized formulation described in Example 1 (Oral: 5 mM PD, 10% DMSO, in PBS; Topical: 1 mM PD, 2% DMSO, 2% HPMC, in PBS) and the tested PD dosage resulted in no toxicity.

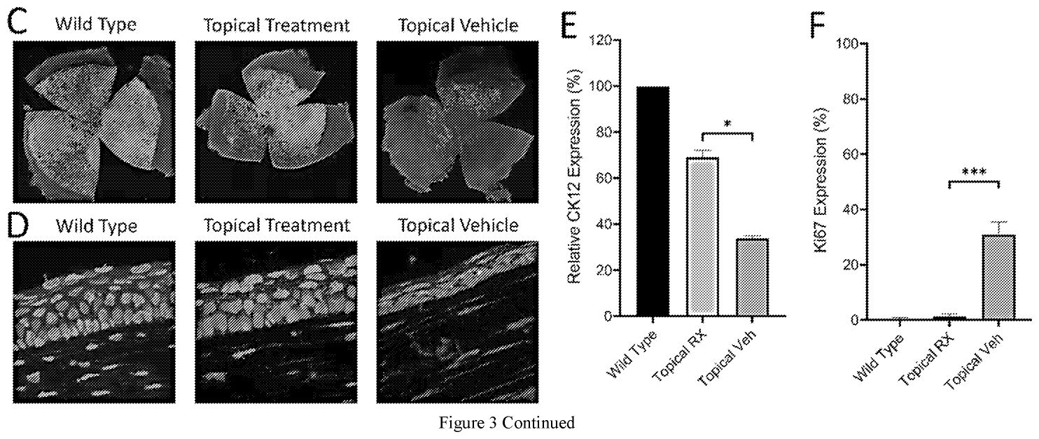

Aniridia mice were treated with or topical PD composition or vehicle, from postnatal day P5 to P30. Wild type (WT) littermates were used as normal reference. OCT and slit lamp images were used to compare the corneal epithelial/stromal thickness, clarity and scarring. H&E and immunostaining with PAX6 and Cytokeratin (CK)12 were used to compare the morphology, protein expression and differentiation. P30 PAX6 staining confirmed increase in PAX6 protein expression in corneas with both oral (2.3±0.2 folds) and topical (2.5±0.1 folds) methods (P<0.001) (oral data not shown, topical data FIG. 3A).

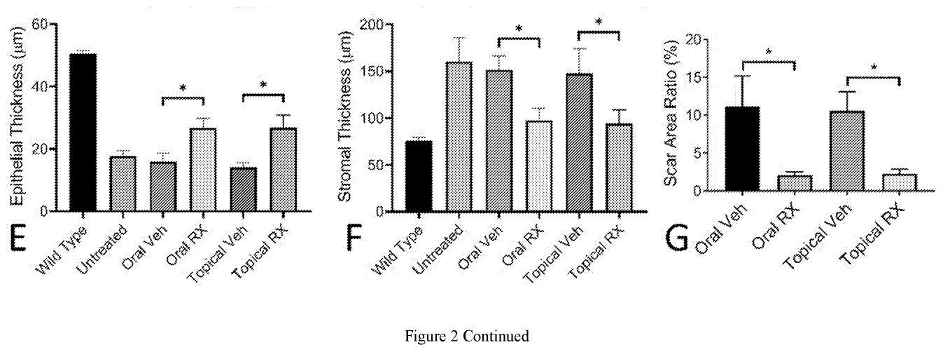

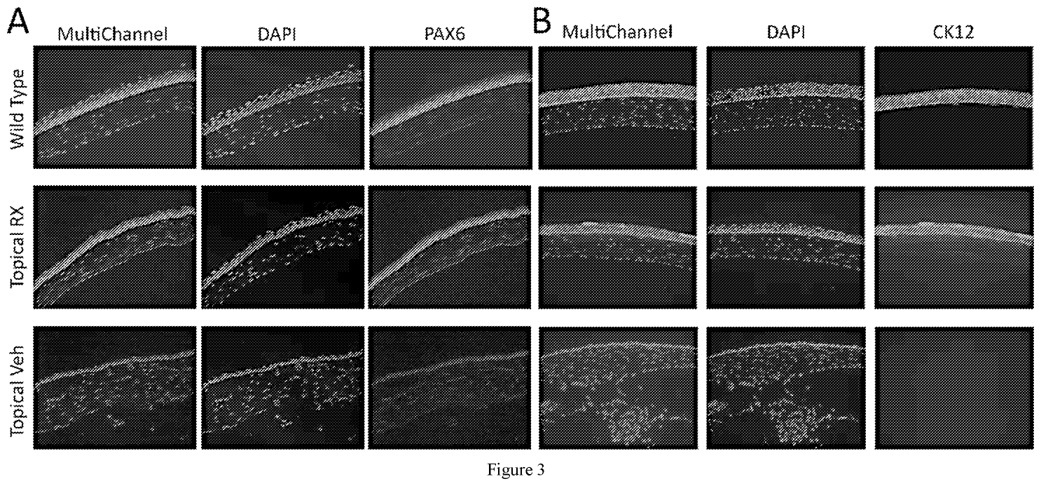

The oral and topical administration of the MEK inhibitor PD resulted in a decrease in the percentage of scar area ratio in the mice suffering from Aniridia. FIG. 2 provides the data from the topical administration of PD compositions. Corneal scarring was significantly less in both oral (2.1±0.4% vs. 11.2±4.0%, P<0.001) and topical (2.3±0.6% vs. 10.6±2.5%, P<0.001) treatment groups compared to vehicle control. The area of scarring was measured manually based on the corneal opacification in the scar area on the slit lamp images using ImageJ (NIH). The ratio of the scar area to total corneal area was reported as a percentage. Corneal stromal thickness at P30 was significantly less than control in both oral (24.7±4.1 μm vs. 15.2±1.5 μm, P<0.001) and topical (25.5±4.7 μm vs. 14.0±1.5 μm, P<0.001) treatment groups (WT: 50.5±1.1 μm). These measurements show the promotion of a more normal differentiation of the corneal epithelium and stroma (FIG. 2). Corneal epithelial and stromal thicknesses were measured at the central cornea and outside the scar area via OCT imaging.

Hematoxylin and eosin (H&E) staining was carried out on Aniridia cornea tissue. The cornea treated with PD showed a more similar histological pattern to normal. FIG. 2 provides representative H&E staining of frozen sections from day 30).

Immunohistochemical staining of the mice eyes for DAPI, PAX6 and CK12 (a marker for normal corneal epithelium) was also carried out. As shown in FIG. 3, immunostaining of the mice eyes after 30 days of treatment showed that topical treatment with PD induced expression of PAX6 protein in the cornea (2.5±0.1 folds compared to control, P<0.001), which led to a more normal differentiation of the corneal epithelium (confirmed with CK12, a specific marker for normal corneal epithelium) (FIG. 3A). P90 corneal whole mount CK12 staining showed better corneal epithelial differentiation in treatment group compared to control (Normal CK12 intensity: 100%; oral: 64±3% vs. 31±6%, P<0.01; topical treatment: 69±32% vs. topical vehicle 34±13%, P<0.05) (FIG. 3C,E).

Better epithelial differentiation leads to a clearer cornea, less scarring, and ultimately better vision. Thus, using this exemplary topical formulation, progression of the severe corneal manifestations in a mouse model of Aniridia was successfully prevented and promoted a clear cornea and improved ocular function.

### Example 3

### Electroretinography Analysis In Aniridia Mouse Model

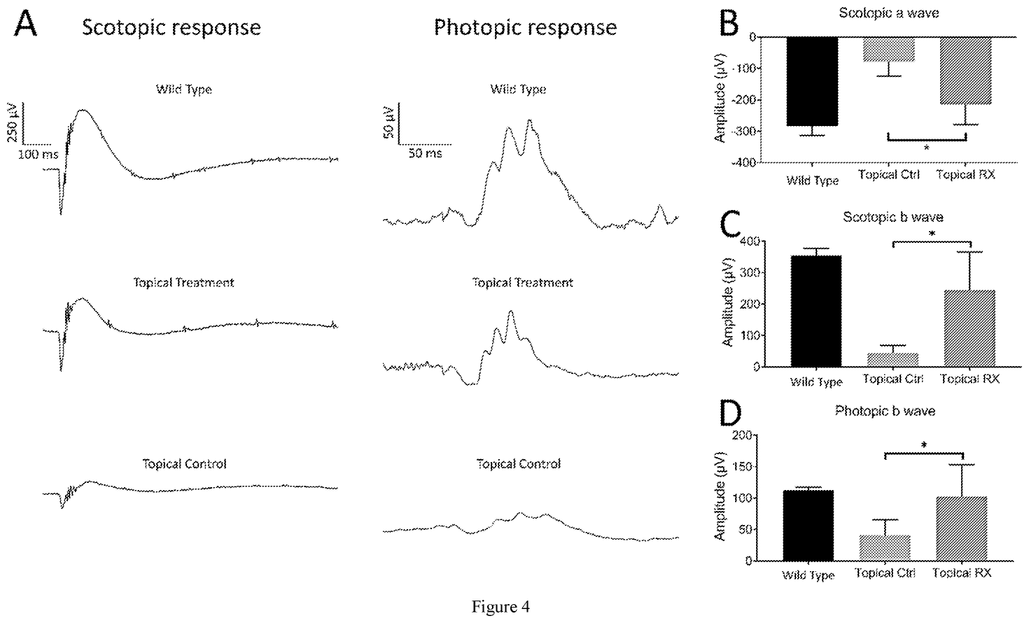

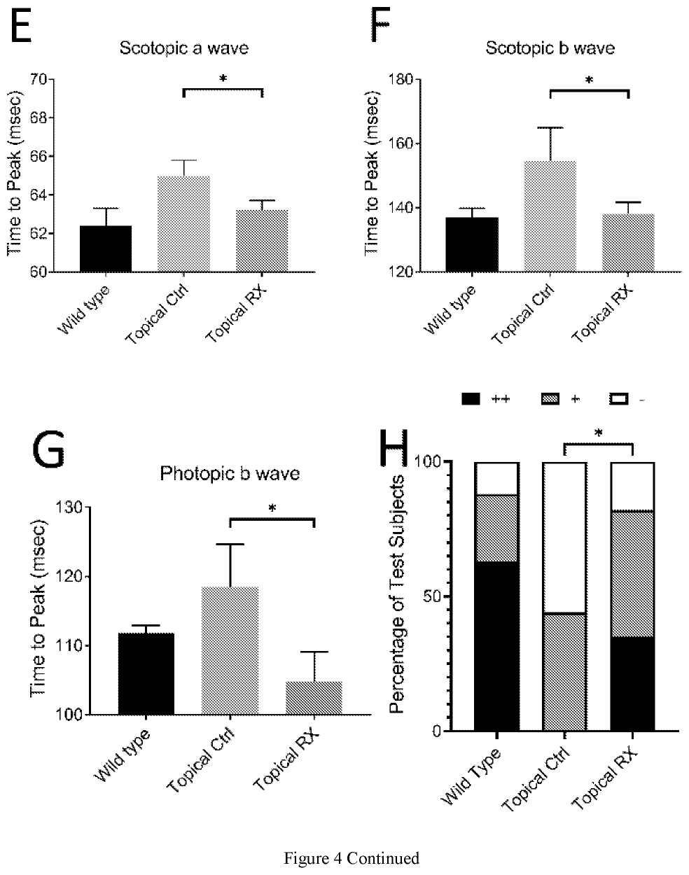

Retina function was assessed in the Aniridia mouse model using P90 electroretinography (ERG). Aniridia mice were treated with the PD composition described in Example 1 or vehicle. P90 electroretinography showed that the treatment group had a significantly better ocular/retinal function compared to the control group. As shown in FIG. 6 and Table 1 below, P90 ERG showed increased a and 6 waves in topical group compared to control (P<0.05). This is demonstrated by a more normal wave shape, and a better maximum intensity of the alpha and beta waves in both scotopic and photopic modalities (FIG. 4).

| | | | |
|---|---|---|---|
| | Topical | | |
| Wild type | vehicle | Topical PD | P value |

Scotopic

| | | | | |
|---|---|---|---|---|
| α wave | −283.6 ± 30.4 | −79.1 ± 45.0 | −214.5 ± 64.1 | 0.008031679 |
| β wave | 354.6 ± 21.9 | 42.4 ± 26.6 | 244.8 ± 120.1 | 0.020133206 |

Photopic

| | | | | |
|---|---|---|---|---|
| β wave | 112.6 ± 4.4 | 40.8 ± 24.8 | 102.1 ± 50.9 | 0.045328557 |

Early postnatal induction of PAX6 can alleviate several manifestations of aniridia. This approach can be used to partially treat and/or prevent aniridia, particularly the corneal disease which is known to be progressive.

### Example 4

### Corneal Injury Model

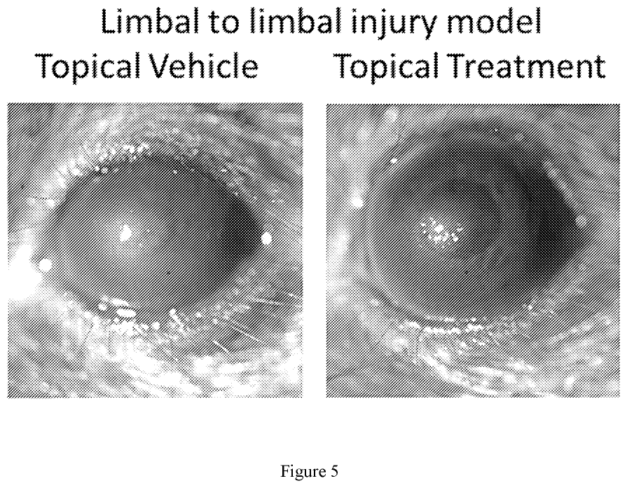

The PD composition provided in Example 1 was tested in an injury model, in which we injured the epithelium of the cornea up to the limbal area, creating a severe scar forming wound with limbal stem cell deficiency. Using an alger brush, the corneal epithelium was scraped under general anesthesia from limbus to limbus to create a limbal stem cell deficiency model. Following this injury, corneal limbal stem cell deficiency happens, similar to human disease, and the cornea becomes scarred, opacified and vascularized by time. As shown in FIG. 5, the PD treatment significantly reduced the corneal opacity and neovascularization in this model as well.

What is claimed:

1. An ophthalmic composition comprising a MAPK/ERK kinase (MEK) inhibitor and phosphate buffered saline (PBS).

2. The ophthalmic composition of claim 1, further comprising about 0.1% to 20% DMSO and about 1% to about 20% HPMC and the at MAPK/ERK kinase (MEK) inhibitor is a concentration of about 0.1 to about 10 μM or about 10% to 50% poloxamer 407 and about 1% to about 20% MEK inhibitor.

3. The ophthalmic composition of claim 1 or 2, further comprising a pharmaceutically acceptable ophthalmic excipient.

4. The ophthalmic composition of claim 3, wherein the ophthalmic excipient is polyvinyl alcohol, povidone, hydroxypropyl methyl cellulose, poloxamers, polyols, Carbopol, pluronics, carbomers, carboxymethyl cellulose, hydroxyethyl cellulose, cyclodextrins, phosphate buffer, citrate buffer, Tris buffer, sodium chloride, potassium chloride, polysorbate 80, vegetable oil, preservative or a combination thereof.

5. The ophthalmic composition of any one of claims 1-4 provided in a suitable carrier, or formulated for topical administration.

6. The ophthalmic composition of any one of claims 1-5, wherein the MAPK/ERK kinase (MEK) inhibitor is a MEK1 or MEK2 inhibitor.

7. The ophthalmic composition of claim 6, wherein the MEK1 or MEK2 inhibitor is PD098059, PD0325901/Mirdametnib, Trametinib, Cobimetinib, MEK 1/2 inhibitor AS703988, MEK inhibitor AZD8330, MEK inhibitor CS3006, MEK inhibitor RO4987655, MEK inhibitor SHR 7390, MEK inhibitor TAK-733, MEK/Aurora kinase dual inhibitor BI 847325, MEK-1/MEKK-1 inhibitor E6201, refametinib, selumetinib, binimetinib, CI-1040, AZD6244, GDC-0973, GSK1120212, AS703026, BS203580, MEK162, GDC-0623, RO5126766, WX-554, HL-085, or U0126.

8. The ophthalmic composition of any one of claims 1-5, wherein the MAPK/ERK kinase (MEK) inhibitor is an ERK inhibitor.

9. The ophthalmic composition of claim 8, wherein the ERK inhibitor is FRI-20, ON-01060, VTX-11e, 25-OH-D3-3-BE, B3CD, bromoacetoxycalcidiol, 180204 AEZ-131, AEZS-131, AEZS-136, SCH-772984, AZ-13767370, BL-EI-001, LY-3214996, LTT-462, KO-947 CC-90003, GDC-0994, RG-7842, MK-8353, SCH900353, BVD-523, or Ulixertinib.

10. The ophthalmic composition of any one of claims 1-5, wherein the MAPK/ERK kinase (MEK) inhibitor is PD0325901.

11. A method of reducing or preventing scarring, opacification, or neovascularization on the surface of the eye of a subject comprising administering to the subject an ophthalmic composition of any one of claims 1-10.

12. A method of improving vision or reducing limitations on vision in a subject in need, wherein the method comprises administering to the subject an ophthalmic composition of any one of claims 1-10.

13. The method of claim 11 or 12, wherein the subject is suffering from Aniridia, corneal scarring, corneal opacification, corneal neovascularization, ocular surface inflammation, severe ocular surface injuries, limbal stem cell deficiency, infections, auto-immune disease, inflammatory diseases or congenital diseases associated with reduced expression of PAX6.

14. A method of treating an ocular disease or condition in a subject in need, wherein the method comprises administering to the subject an ophthalmic composition of any one of claims 1-10.

15. The method of claim 14 wherein the ocular disease or condition is Aniridia, corneal scarring, corneal opacification, corneal neovascularization, ocular surface inflammation, severe ocular surface injuries, limbal stem cell deficiency, infections, auto-immune disease, inflammatory diseases or congenital diseases associated with reduced expression of PAX6.

16. A method of inducing PAX6 expression in a cell comprising administering to the cell an effective amount of a MAPK/ERK kinase (MEK) inhibitor.

17. The method of claim 16, wherein the cell is an epithelial cell of the surface of the eye or a fibroblast cell on the surface of the eye.

18. A method of reducing or preventing scarring, opacification, or neovascularization on the surface of the eye of a subject comprising administering to the subject an effective amount of a MAPK/ERK kinase (MEK) inhibitor.

19. A method of improving vision or reducing limitations on vision in a subject in need, wherein the method comprises administering to the subject an effective amount of a MAPK/ERK kinase (MEK) inhibitor.

20. The method of claim 18 or 19, wherein the subject is suffering from Aniridia, corneal scarring, corneal opacification, corneal neovascularization, ocular surface inflammation, severe ocular surface injuries, limbal stem cell deficiency, cancer, infections, auto-immune disease, inflammatory diseases or congenital diseases associated with reduced expression of PAX6.

21. A method of treating an ocular disease or condition in a subject in need, wherein the method comprises administering to neovascularization the subject an effective amount of a MAPK/ERK kinase (MEK) inhibitor.

22. The method of claim 21 wherein the ocular disease or condition is Aniridia, scarring on the surface of the eye, opacification of the surface of the eye, neovascularization on the surface of the eye, ocular surface inflammation, severe ocular surface injuries, limbal stem cell deficiency, cancer, infections, auto-immune disease, inflammatory diseases or congenital diseases associated with reduced expression of PAX6.

23. The method of any one of claims 16-22, wherein the MAPK/ERK kinase (MEK) inhibitor is a MEK1 or MEK2 inhibitor.

24. The method of claim 23, wherein the MEK1 or MEK2 inhibitor is PD098059, PD0325901, Tratetinib, Cobimetinib, MEK 1/2 inhibitor AS703988, MEK inhibitor PD325089, MEK inhibitor AZD8330, MEK inhibitor CS3006, MEK inhibitor RO4987655, MEK inhibitor SHR 7390, MEK inhibitor TAK-733, MEK/Aurora kinase dual inhibitor BI 847325, MEK-1/MEKK-1 inhibitor E6201, refametinib, selumetinib, CI-1040, AZD6244, GDC-0973, GSK1120212, AZD8330, RO51267655, RO4987655, AS703026, BS203580, MEK162, GDC-0623, RO5126766, WX-554, HL-085, or U0126.

25. The method of any one of claims 16-22, wherein the MAPK/ERK kinase (MEK) inhibitor is an ERK inhibitor.

26. The method of claim 25, wherein the ERK inhibitor is FRI-20, ON-01060, VTX-11e, 25-OH-D3-3-BE, B3CD, bromoacetoxycalcidiol, 180204 AEZ-131, AEZS-131, AEZS-136, SCH-772984, AZ-13767370, BL-EI-001, LY-3214996, LTT-462, KO-947 CC-90003, GDC-0994, RG-7842, MK-8353, SCH900353, BVD-523, or Ulixertinib.

27. The method of any one of claims 16-22, wherein the MAPK/ERK kinase (MEK) inhibitor is PD0325901, at a concentration of about 0.1 to about 10 μm.

28. The method of any one of claims 16-27, wherein the MAPK/ERK kinase (MEK) inhibitor is administered as an ophthalmic composition, and the composition comprises the MAPK/ERK kinase (MEK) inhibitor and PBS.

29. The method of claim 28, wherein the ophthalmic composition further comprises about 1% to about 20% DMSO and about 1% to about 20% HPMC.

30. The method of claim 28, where the ophthalmic composition further comprises about 10% to 50% poloxamer 407.

31. The method of any one of claims 28-30, wherein the MAPK/ERK kinase (MEK) inhibitor is at a concentration of about 0.1 to about 10 μM.

32. The method of any one of claims 16-31, wherein the MAPK/ERK kinase (MEK) inhibitor is administered by topical route or oral route.

33. Use of an effective amount of a MAPK/ERK kinase (MEK) inhibitor for the preparation of a medicament for inducing PAX6 expression in a cell comprising administering to the cell an effective amount of a MAPK/ERK kinase (MEK) inhibitor.

34. The use of claim 33, wherein the cell is an epithelial cell of the surface of the eye or a fibroblast cell on the surface of the eye corneal epithelial cell.

35. Use of an effective amount of a MAPK/ERK kinase (MEK) inhibitor for the preparation of a medicament for the reducing or preventing scarring, opacification, or neovascularization on the surface of the eye of a subject.

36. Use of an effective amount of a MAPK/ERK kinase (MEK) inhibitor for the preparation of a medicament for improving vision or reducing limitations on vision in a subject in need, wherein the method comprises administering to the subject.

37. The use of claim 35 or 36, wherein the subject is suffering from Aniridia, scarring on the surface of the eye, corneal opacification, neovascularization on the surface on the eye, ocular surface inflammation, severe ocular surface injuries, limbal stem cell deficiency, cancer, infections, auto-immune disease, inflammatory diseases or congenital diseases associated with reduced expression of PAX6.

38. Use of an effective amount of a MAPK/ERK kinase (MEK) inhibitor for the preparation of a medicament for treating an ocular disease or condition in a subject in need.

39. The use of claim 38 wherein the ocular disease or condition is Aniridia, scarring on the surface of the eye, corneal opacification, neovascularization on the surface of the eye, ocular surface inflammation, severe ocular surface injuries, limbal stem cell deficiency, cancer, infections, auto-immune disease, inflammatory diseases or congenital diseases associated with reduced expression of PAX6.

40. The use of any one of claims 33-39, wherein the MAPK/ERK kinase (MEK) inhibitor is a MEK1 or MEK2 inhibitor.

41. The use of claim 40, wherein the MEK1 or MEK2 is PD098059, PD0325901, Tratetinib, Cobimetinib, MEK 1/2 inhibitor AS703988, MEK inhibitor PD325089, MEK inhibitor AZD8330, MEK inhibitor CS3006, MEK inhibitor RO4987655, MEK inhibitor SHR 7390, MEK inhibitor TAK-733, MEK/Aurora kinase dual inhibitor BI 847325, MEK-1/MEKK-1 inhibitor E6201, refametinib, selumetinib, CI-1040, AZD6244, GDC-0973, GSK1120212, AZD8330, RO51267655, RO4987655, AS703026, BS203580, MEK162, GDC-0623, RO5126766, WX-554, HL-085, or U0126.

42. The use of any one of claims 33-39, wherein the MAPK/ERK kinase (MEK) inhibitor is an ERK inhibitor.

43. The use of claim 42, wherein the ERK inhibitor is FRI-20, ON-01060, VTX-11e, 25-OH-D3-3-BE, B3CD, bromoacetoxycalcidiol, 180204 AEZ-131, AEZS-131, AEZS-136, SCH-772984, AZ-13767370, BL-EI-001, LY-3214996, LTT-462, KO-947 CC-90003, GDC-0994, RG-7842, MK-8353, SCH900353, BVD-523, or Ulixertinib.

44. The use of any one of claims 33-39, wherein the MAPK/ERK kinase (MEK) inhibitor is PD0325901 at a concentration of about 0.1 to about 10 μm.

45. The use of any one of claims 33-44, wherein the medicament is administered as an ophthalmic composition, and the composition comprises the MAPK/ERK kinase (MEK) inhibitor and PBS.

46. The use of claim 45, wherein the ophthalmic composition further comprises about 1% to about 20% DMSO and about 1% to about 20% HPMC.

47. The use of claim 46, where the ophthalmic composition further comprises about 10% to 50% poloxamer 407.

48. The use of any one of claims 45-47, wherein the MAPK/ERK kinase (MEK) inhibitor is at a concentration of about 0.1 to about 10 μM.

49. The use of any one of claims 33-48, wherein the medicament is formulated for administration by topical route or oral route.

50. A composition for inducing PAX6 expression in a cell, wherein the composition comprises an effective amount of a MAPK/ERK kinase (MEK) inhibitor.

51. The composition of claim 50, wherein the cell is an epithelial cell of the surface of the eye or a fibroblast cell on the surface of the eye corneal epithelial cell corneal epithelial cell.

52. A composition for reducing or preventing scarring on the surface of the eye, opacification, or neovascularization on the surface of the eye of a subject, wherein the composition comprises an effective amount of a MAPK/ERK kinase (MEK) inhibitor.

53. A composition for improving vision or reducing limitations on vision in a subject in need, wherein the composition comprises an effective amount of a MAPK/ERK kinase (MEK) inhibitor.

54. The composition of claim 52 or 53, wherein the subject is suffering from Aniridia, scarring on the surface of the eye, corneal opacification, neovascularization on the surface of the eye, ocular surface inflammation, severe ocular surface injuries, cancer, limbal stem cell deficiency infections, auto-immune disease, inflammatory diseases or congenital diseases associated with reduced expression of PAX6.

55. A composition for of treating an ocular disease or condition in a subject in need, wherein the composition comprises an effective amount of a MAPK/ERK kinase (MEK) inhibitor.

56. The composition of claim 55 wherein the ocular disease or condition is Aniridia, scarring on the surface of the eye, corneal opacification, neovascularization on the surface of the eye, ocular surface inflammation, severe ocular surface injuries, limbal stem cell deficiency, cancer, infections, auto-immune disease, inflammatory diseases or congenital diseases associated with reduced expression of PAX6.

57. The composition of any one of claims 50-56, wherein the MAPK/ERK kinase (MEK) inhibitor is a MEK1 or MEK2 inhibitor.

58. The composition of claim 57, wherein the MEK1 or MEK2 inhibitor is PD098059, PD0325901, Tratetinib, Cobimetinib, MEK 1/2 inhibitor AS703988, MEK inhibitor PD325089, MEK inhibitor AZD8330, MEK inhibitor CS3006, MEK inhibitor RO4987655, MEK inhibitor SHR 7390, MEK inhibitor TAK-733, MEK/Aurora kinase dual inhibitor BI 847325, MEK-1/MEKK-1 inhibitor E6201, refametinib, selumetinib, CI-1040, AZD6244, GDC-0973, GSK1120212, AZD8330, RO51267655, RO4987655, AS703026, BS203580, MEK162, GDC-0623, RO5126766, WX-554, HL-085, or U0126.

59. The composition of any one of claims 50-56, wherein the MAPK/ERK kinase (MEK) inhibitor is an ERK inhibitor.

60. The composition of claim 59, wherein the ERK inhibitor is FRI-20, ON-01060, VTX-11e, 25-OH-D3-3-BE, B3CD, bromoacetoxycalcidiol, 180204 AEZ-131, AEZS-131, AEZS-136, SCH-772984, AZ-13767370, BL-EI-001, LY-3214996, LTT-462, KO-947 CC-90003, GDC-0994, RG-7842, MK-8353, SCH900353, BVD-523, or Ulixertinib.

61. The composition of any one of claims 50-60, wherein the MAPK/ERK kinase (MEK) inhibitor is PD0325901 at a concentration of about 0.1 to about 10 μm.

62. The composition of any one of claims 50-61, is an ophthalmic composition, and the composition comprises the MAPK/ERK kinase (MEK) inhibitor and PBS.

63. The composition of claim 62, wherein the ophthalmic composition further comprises about 1% to about 20% DMSO and about 1% to about 20% HPMC.

64. The method of claim 62, where the ophthalmic composition further comprises about 10% to 50% poloxamer 407.

65. The composition of any one of claims 62-64, the MAPK/ERK kinase (MEK) inhibitor is at a concentration of about 0.1 to about 10 μM.

66. The composition of any one of claims 50-65, wherein the composition is formulated for the administration by topical route or oral route.
